(19) United States
(12) Patent Application Publication
(10) Pub. No.: US 2022/0135664 A1
(43) Pub. Date: May 5, 2022

(54) Use of an antibody and antisense in the treatment of Congenital Muscular Dystrophy

(71) Applicant: University of Zagreb School of Medicine, Zagreb (HR)

(72) Inventors: Slobodan Vukicevic, Zagreb (HR); Kuber T. Sampath, Holliston, MA (US)

(21) Appl. No.: 17/453,305

(22) Filed: Nov. 2, 2021

Related U.S. Application Data
(60) Provisional application No. 63/108,442, filed on Nov. 2, 2020.

(51) Int. Cl. C07K 16/22; A61P 21/00

(52) U.S. Cl. CPC C07K 16/22; A61K 2039/545; C07K 2317/24; A61P 21/00

(57) ABSTRACT

The present invention relates to a pharmaceutical composition comprising an antibody that binds to BMP1.3 non-catalytic domain that contains either CUB-3 or CUB-4 or both CUB-3 and CUB-4 or EGF-2 or both CUB-3 and CUB-4 and EGF-2 and at the C-terminal region for use in prevention and/or therapeutic treatment of muscular dystrophy.

### FIELD OF THE INVENTION

The present invention relates to a pharmaceutical composition for the delaying the progression and/or treatment of Congenital Muscular Dystrophy, a rare neuromuscular genetic disorder. More particularly, the invention relates to the pharmaceutical composition of therapeutics as treatment to improve skeletal muscle mass, mobility, muscle-nerve coordination and survival. The composition of biologics referred include are antibodies and antisense that may be used as therapeutics for congenital muscular dystrophy, including Duchenne Muscular Dystrophy.

### BACKGROUND OF THE INVENTION

The structural identity of NAS39 and the Bmp1 gene provides evidence of an evolutionary core pathway conservation from C. elegans, which has no major organs, towards mammalian organ specialization with newly developed regulatory pathway of Bmp1 alternatively spliced variants (isoforms). Rather than preserving 40 astacin-like genes in C. elegans for multiple functions, they evolved in mammals into fewer Bmp1/Tolloid genes.

BMP1, a zinc-dependent metalloproteinase, comprises a promiscuous astacin-like catalytic domain followed by several CUB (for complement C1r/C1s, uEGF, BMP-1) and EGF domains (FIG. 1). The regions of identified peptides from BMP1.3 gene isoform is indicated by * in FIG. 1. Proteins were identified by liquid chromatography-mass spectrometry (LC-MS) using the Mascot search engine. Peptides with a mass deviation<10 ppm were accepted, and at least two detected peptides were required for protein identification. It was originally identified from bovine bone matrix as a “contaminant” in the osteogenic fractions capable of inducing new bone formation at ectopic sites. Subsequently, it has been shown to be involved in the processing of major and minor fibrillar collagens at the C-terminal region and pro-lysyl oxidases into active enzymes to assemble collagen fibrils.

BMP-1.3 is a variant of BMP1 and has two additional CUB and one additional EGF domain (FIG. 1). BMP1.3 is evolutionarily conserved and structurally related to mammalian Tolloid-like metalloproteinase (mTLD). BMP1.3 has been shown to be involved in the processing of 1) collagens (I-III), 2) small leucin-rich proteoglycans, SLRPs (decorin, bigycan), 3) disruption of basement membrane mesh-work (laminin, collagen VI and VII, perlecan), 4) activation of release of TGF-β from latent binding protein and promoting the degradation of TGF-β antagonists (soluble β glycan and CD-109 and SLRPs), 5) degradation of TGF-β antagonists (soluble β glycan and CD-109 and SLRPs) and 6) activation of TGF-β superfamily proteins precursor proteins into active mature forms (BMP-2/4, GDF-8 (myostatin) and GDF-11, and processing Chordin, a BMP antagonist.

The Congenital Muscular Dystrophy (CMD) represents a group of rare neuromuscular dystrophies (MDs) traditionally defined as having symptom right from the onset at birth or in the first 2 years of life. CMDs are distinct from congenital myopathies, which are characterized by different pathologic features and specific genetic etiologies (e.g., Duchenne Muscular Dystrophy, DMD). Three major categories of CMDs are commonly recognized, each of which has distinct, well described phenotypic features: 1) Collagenopathies (also known as collagen VI related myopathies), 2) Merosinopathies (also known as merosin deficient CMDs [MDCs], laminin a2 [LAMA2]-related CMDs, and MDC1A), and 3) Dystroglycanopathies (also known as a-dystroglycan-related MDs), including Fukuyama CMD, muscle-eye-brain disease, and Walker-Warburg syndrome. Other rare CMDs do not fit into any of the classic categories. In European populations, the prevalence is in the range of 4 in 500,000 people, with MDC1A counting for 34% of all CMDs.

The Laminin-deficient CMD type 1A (MDC1A) is an autosomal recessive disease caused by mutations in the lama2 gene that encodes for the alpha 2 chain of the muscle and Schwann cell specific heterotrimeric extracellular matrix protein Laminin-211. Absence of a functional copy of this protein results in defective myofiber anchoring and dysregulation, including failed regeneration, inflammation, fibrosis, apoptosis, necrosis and failed regeneration. Children with this disease present soon after birth with severe weakness, atrophy and hypotonia and die prematurely due to respiratory complications or failure to thrive. The Lama2^{DyW-/+} (DyW) mouse is the most commonly study animal model for MDC1A research. Magnetic resonance imaging (MRI) tightly corresponds with pathological changes in DyW mice being a variable and effective non-invasive tool for assessing pathological changes. Unlike the mdx mouse model frequently utilized for Duchenne Muscular Dystrophy in DMD research, DyW mice have a more severe phenotype and rarely survive past one to two months.

Laminin-211 is primarily expressed in the basal membranes of skeletal muscle and Schwann cells, in capillaries between astrocyte foot processes and vessels of the brain. Other tissues include heart, kidney, lung, stomach, placenta, and testes. It is expressed as early as week 7 in human embryos and E11 in mice. An important driver of fibrosis in this disease is TGF-β. The very early onset of upregulated TGF-β with Smad2/3 is similar in MDC1A patients and DyW mice, supporting the translatability of this similar finding. There is also a parallel downregulation of inhibitor Smad7, suggesting even further amplification of the TGFβ signaling pathway. Downstream genes encoding ECM proteins are also upregulated, including fibronectin, osteopontin, periostin, and collagen I. There is chronically dysregulated matrix remodeling process that is etiological to the pathology following loss of laminin-alpha 2. Treatment with laminin-111 and laminin-211 of immune deficient DyW mice resulted in improved muscle function and pathology, but was not efficacious on muscle regeneration.

Muscle fibers are surrounded by basement membrane (BM) via muscle plasma membrane called sarcolemma, while BM is crucial for muscle fiber stability and signal transduction. LAMA2 MD is among the most frequent CMDs in Europe. Patients with complete absence of laminin-α2 are hypotonic at birth, fail to ambulate and succumb to respiratory complications. Patients with less severe symptoms often express low amounts of lam inin-alpha2 or a truncated form. Most widely used mouse model are DyW mice that show very early onset muscular dystrophy and a markedly shortened life span. A less severe model dy2J/dy2J mice express an N-terminally truncated mutant of laminin-alpha2, showing a mild dystrophic pathology. It has been demonstrated that defects in BM assembly and its connection to the sarcolemma are the primary cause of LAMA2-CMD.

There is no drug available for MDC1A. Most strategies used to alleviate fibrosis directly or indirectly by targeting TGF-β signaling pathway. Losartan, an approved AT1R blocker routinely used to control hypertension, showed remarkable amelioration of fibrosis in mouse models of MDC1A, as well as anti-inflammatory effects. Losartan, however, does not lead to increased body or muscle weight in DyW mice. Therapy with chemically modified derivative of Losartan (DuP-553; Merck) also showed improved locomotor activity. A pharmaceutical formulation of the Ang 1-7 peptide (TXA127) has been granted Orphan Drug designation for muscular dystrophies to Context Therapeutics to initiate clinical trials. Some effects in preclinical models of MDC1A have been observed by inhibition of proapoptotic protein Bax, as well as inhibition of GAPDH-Siah1 mediated apoptosis with Omigapil.

Recently, we have shown that BMP1.3 is available in circulation and credentialing that its levels are elevated in patients with chronic kidney failure, hepatic fibrosis and acute myocardial infarction. Subsequently, we demonstrated the translatability of this target in that systemic administration of anti-BMP1.3 antibody ameliorated the kidney, liver and heart function and extended the survival of animals in these diseases. The discovery that BMP1.3 circulates in the blood and that its level is elevated in parenchymal fibrosis patients suggests that it might have multiple, yet unpredicted functions in orchestrating the extracellular matrix (ECM) assembly and regulating latent growth factors activity locally and systemically, and thus affects the pathophysiology of major human diseases.

It has been shown that BMP1.3 is readily available in plasma and elevated by several-fold in patients with chronic kidney disease (CKD), acute myocardial infarction (AMI) and hepatic fibrosis (HF). We also discovered that BMP1.3 mRNA transcript was elevated in the skeletal muscle and organs of congenital muscular dystrophy mice (DyW mice) starting at 2 weeks of age and then increased with severity of the diseases. Therefore, BMP1.3 may have a critical role in the progression of the disassembly of cell membrane-muscle fiber-extracellular matrix network associated with congenital muscular dystrophy (CMD).

As BMP1.3 transcript, and therefore protein, is strongly elevated in CMD mice (DyW), we examined the effects of anti-BMP1.3 antibody. The results showed surprisingly an improvement in the mobility and overall motor-coordination, increased grip activity, increased muscle mass and an improved survival. Furthermore, the transcripts of BMP1.1, BMP1.3 and TGF-ß1 levels were reduced in the skeletal muscle, kidney and liver of DyW CKD mice after receiving anti-BMP1.3 antibody.

### SUMMARY OF THE INVENTION

The present invention provides new methods of treatment based on discoveries relating to the circulation of BMP-1.3 in the blood of individuals. Moreover, the discoveries disclosed herein have led to the development of new treatment methods which enhance the effects of anti-BMP1.3 antibody in individuals suffering from particular muscular dystrophies. For the first time, it has been observed that BMP1.3 gene expression was highly enhanced in the skeletal muscles of an individual with congenital muscular dystrophy (CMD). Administration of anti-BMP1.3 antibody was discovered to improve mobility and provide increased muscle volume. Therefore, BMP1.3 inhibition is a novel therapeutic strategy for use in treatments for reversing the progression of CMD.

One embodiment of the present invention involves a method of treating and delaying the progression of muscular dystrophy comprising of administering to an individual in need thereof a pharmaceutical composition comprising an antibody that binds to BMP1.3 non-catalytic domain that contains either CUB-3 or CUB-4 or both CUB-3 and CUB-4 or EGF-2 or both CUB-3 and CUB-4 and EGF-2 and at the C-terminal region.

Muscular dystrophy is selected from the group consisting of congenital muscular dystrophy (CMD), Duchenne muscular dystrophy (DMD), Myotonic muscular dystrophy, Becker muscular dystrophy, Limb-girdle muscular dystrophy, Facioscapulohumeral muscular dystrophy, Oculopharyngeal muscular dystrophy, Distal muscular dystrophy, and Emery-Dreifuss muscular dystrophy. The Congenital Muscular Dystrophy (CMD) is associated with Collagenopathies (also known as collagen VI related myopathies), Merosinopathies (also known as merosin deficient CMDs [MDCs], laminin a2 [LAMA2]-related CMDs, and MDC1A); and (3) Dystroglycanopathies (also known as a-dystroglycan-related MDs), including Fukuyama CMD, muscle-eye-brain disease, and Walker-Warburg syndrome.

A further embodiment of the present invention is to provide an antibody that binds to BMP1.3 non-catalytic domain that contain either CUB 3 or CUB4 or both CUB 3 and CUB4 or EGF-2 at the C-terminal region that is a humanized antibody.

A further embodiment of the present invention provides a method of producing an antibody that binds to BMP1.3 non-catalytic domain that contains either CUB-3 or CUB-4 or both CUB-3 and CUB-4 or EGF-2 or both CUB-3 and CUB-4 and EGF-2 and at the C-terminal region.

A further embodiment of the present invention provides a pharmaceutical composition comprising an antibody that binds to BMP1.3 non-catalytic domain that contains either CUB-3 or CUB-4 or both CUB-3 and CUB-4 or EGF-2 or both CUB-3 and CUB-4 and EGF-2 and at the C-terminal region for use in treating and delaying the progression of muscular dystrophy in an individual.

In particular, the present invention provides an anti-fibrosis biotherapeutic for use in the first stage for congenital muscular dystrophy.

### BRIEF DESCRIPTION OF THE DRAWINGS

FIG. 1 shows a discovery and characterization of BMP1.3 gene isoform in the plasma of healthy individuals and patients with CKD (−), LD (−), CMD and AMI.

FIG. 2 illustrates DyW−/−mouse without treatment (FIGS. 2A, 2B) with affected dystrophy leg (red arrow), treatment with polyclonal 50 μg anti-BMP1.3 (FIG. 2C), 2 h after 3^{rd} treatment (FIG. 2D), compared WT mouse (green arrow) and treated DyW−/− (red arrow) in 5^{th} week of life (FIG. 2E); treated DyW−/− in 5^{th} week of life (FIG. 2F). The mouse carrying homozygous mutation for laminin α2 gene (Lama2 Dy-W) is smaller in size when compared with the wild type sibling, less active, and has impaired motion. After treatment with polyclonal anti-BMP1.3 antibody (50 μg/kg), its activity was improved after the 1^{st} dose, and mobility was even more improved after subsequent two doses, and it lived for 5 weeks, whereas homozygous mice usually die at 4 weeks, which is a life extension of 20% with this arbitrary therapeutic approach.

FIG. 3 is a collection of graphs illustrating gene expression of BMP1.1, BMP1.3, BMP2, BMP5, TGFβ, Smad2, Smad3 and Smad4 in various organs (liver, muscle, kidney) of DyW mice at 5 weeks of age. Expression is markedly upregulated in DyW mice and is reversed after receiving anti-BMP1.3 antibodies. Values are presented as mean±SD. *P<0.05, **P<0.01.

FIG. 4 is two graphs, illustrating Expression of Atrogin-1 mRNA in C2C12 mouse myoblasts upon atrophy induced by 10 μM (FIG. 4A) and 100 μM (FIG. 4B) dexamethasone and treatment with 10 μg/mL and 20 μg/mL anti-BMP1.3 polyclonal antibody.

FIG. 5 is a collection of images showing collagen detection performed by Sirius red staining on muscle sections from heterozygous mice (DyW+/−), homozygous mice (DyW−/−) and homozygous DyW mice treated with anti BMP1-3 antibody (50 μg/kg); scale bare indicates 50 μm in a) and 100 μm in b).

FIG. 6 is graph illustrating Levels of BMP1.3 in serum from healthy subjects and patients affected with AMI determined by an ELISA assay.

FIG. 7 illustrates BMP1.3 polyclonal antibody reduced fibrosis and preserved ejection fraction in rodent models of AMI.

### DETAILED DESCRIPTION OF THE INVENTION

Definitions

The following terms are intended to have the meanings presented therewith below and are useful in understanding the description and intended scope of the present invention.

“Circulate” and “circulating” describe anything that travels or is otherwise transported through the vascular system of an individual.

“Antibody” or “antibody molecule”, as used and understood herein, refers to a specific binding member that is a protein molecule or portion thereof or any other molecule, whether produced naturally, synthetically, or semi-synthetically, which possesses an antigenic binding domain formed by an immunoglobulin variable light chain region or domain (VL) or portion thereof, an immunoglobulin variable heavy chain region or domain (VH) or portion thereof, or a combination thereof. The term “antibody” also covers any polypeptide or protein molecule that has an antigen-binding domain that is identical, or homologous to, an antigen-binding domain of an immunoglobulin. Antibodies may be “polyclonal”, i.e., a population of antigen-binding molecules that bind to different sites on the antigen, or “monoclonal”, i.e., a population of identical antigen-binding molecules that bind to only one site on an antigen. Examples of an antibody molecule, as used and understood herein, include any of the well-known classes of immunoglobulins (e.g., IgG, IgM, IgA, IgE, IgD) and their isotypes; fragments of immunoglobulins that comprise an antigen binding domain, such as Fab or F(ab′)2 molecules; single chain antibody (scFv) molecules; double scFv molecules; single domain antibody (dAb) molecules; Fd molecules; diabody molecules; and fusion proteins comprising such molecules. Diabodies are formed by association of two diabody monomers, which form a dimer that contains two complete antigen binding domains wherein each binding domain is itself formed by the intermolecular association of a region from each of the two monomers (see, e.g., Holliger et al., Proc. Natl. Acad. Sci. USA, 90: 6444-6448 (1993)). Use of such antibody molecules offers the vast array of antibody detection systems and formats available in the art that may be adapted to selectively detect particular BMP-1 isoforms in mixtures, including whole blood, plasma, serum, and various tissue extracts. Examples of formats for using antibody molecules to detect BMP-1 isoforms may include, but are not limited to, immunoblotting (e.g., Western blots, dot blots), immunoprecipitations, affinity methods, immunochips, and the like. Any of variety methods known in the art may be employed to produce antibody molecules to a specific BMP-1 isoform or a portion thereof comprising at least one epitope (antibody binding site) of the BMP-1 isoform.

The terms “disorder” and “disease” are synonymous and refer to any pathological condition, irrespective of cause or etiological agent. A “defect” in a tissue refers to a site of abnormal or deficient tissue growth. A “disease” or “disorder” may be characterized by one or more “defects” in one or more tissues.

As used herein, the terms “treatment” and “treating” refer to any regimen that alleviates one or more symptoms or manifestations of a disease or disorder, that inhibits progression of a disease or disorder, that arrests progression or reverses progression (causes regression) of a disease or disorder, or that prevents onset of a disease or disorder. Treatment includes prophylaxis and includes but does not require cure of a disease or disorder.

A “therapeutically effective amount” is an amount of a compound which inhibits, totally or partially, the progression of the condition, which alleviates, at least partially, one or more symptoms of the disorder, or which enhances or catalyzes the therapeutic or otherwise beneficial effects of another compound. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art.

A composition or method described herein as “comprising” one or more named elements or steps is open-ended, meaning that the named elements or steps are essential, but other elements or steps may be added within the scope of the composition or method. To avoid prolixity, it is also understood that any composition or method described herein as “comprising” (or “which comprises”) one or more named elements or steps also describes the corresponding, more limited, composition or method “consisting essentially of” (or “which consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and close-ended composition or method “consisting of” (or “which consists of”) the named elements or steps to the exclusion of any other unnamed element or step. In any composition or method disclosed herein, known or disclosed equivalents of any named essential element or step may be substituted for that element or step.

Unless indicated otherwise, the meaning of other terms is the same as understood and used by persons in the art, including the fields of medicine, biochemistry, molecular biology, and tissue regeneration.

BMP1.3 is evolutionarily conserved and structurally related to mammalian Tolloid-like metalloproteinase (mTLD). It activates BMP2/4, GDF-8/Myostatin, GDF-11 and TGF-β from latent binding protein and promotes the degradation of several TGF-β antagonists. It is involved in the processing of extracellular matrix proteins: collagens, small leucin-rich proteoglycans, and basement membrane proteins. Recently, we have shown that BMP1.3 is available in circulation and its levels are elevated in patients with chronic kidney failure, hepatic fibrosis and acute myocardial infarction. Administration of anti-BMP1.3 antibody ameliorated the kidney, liver and heart function and extended the survival of animals in these diseases. For the first time, we observed BMP1.3 gene expression was highly enhanced in the skeletal muscles of mice with congenital muscular dystrophy (CMD), while administration of anti-BMP1.3 antibody improved the mobility, increased muscle volume and survival of CMD-DyW mice. We claim that BMP1.3 inhibition represents a novel therapeutic strategy in reversing the progression of CMD.

BMP1.3 represents a core pathway protein essential in fibrosis and inflammation and inhibiting it may limit fibrosis progression. BMP1.3 seems to have an important role in a number of different organs, including kidney, liver, heart and the peripheral muscle.

The discovery that BMP1.3 circulates in the blood and that its level is elevated in parenchymal fibrosis patients suggests that it might have multiple, yet unknown functions in orchestrating the extracellular matrix (ECM) assembly and regulating latent growth factors activity locally and systemically, and thus affects the pathophysiology of major human diseases. We have discovered that inhibition of BMP1.3 has therapeutic benefits for prevention and treatment of in fibrotic diseases, including chronic kidney disease (5/6 nephrectomized (Nx) rats), hepatic fibrosis, and acute myocardic infarction (cardiac ischemia and reperfusion). Inhibition of BMP1.3 has shown a significant effect in prevention of loss of function, and restoration of function where already lost. Evaluation of the effects of anti-BMP1.3 antibodies showed it has substantially reduced renal interstitial and cardiac muscle fibrosis and improved its function, respectively. It is worth to mention that BMP1.3 level is elevated 3-fold in patients with CKD and AMI as compared to age matched healthy individuals. Unexpectedly high BMP1.3 expression initially in skeletal muscles of CMD (DyW) mice prompted us to further explore the therapeutic value of the BMP1.3 inhibition and discovered unexpected recovery of the animals.

Based on preliminary results of anti BMP1.3 antibody in DyW mice and previously observed findings in fibrotic rodent disease models it has been demonstrated that BMP1.3 inhibition may serve as a basis for therapeutic human antibody the development and for clinical evaluation for patients with MDC1A-CMD, a rare neuromuscular disease.

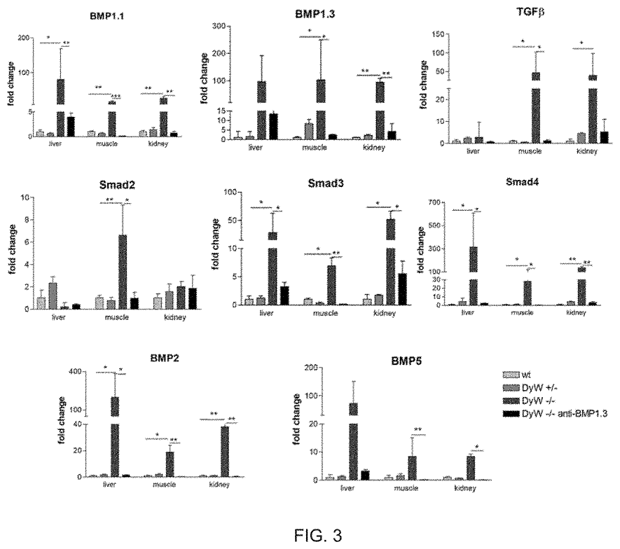

The invention is based on the discovery that BMP1.3 expression was increased in the muscles of DyW mice that has a mutation in laminin 2 gene, the most commonly studied animal model for congenital muscular dystrophy as illustrated in FIG. 2. FIG. 2 shows DyW−/−mouse without treatment (FIGS. 2A, 2B) with affected dystrophy leg (red arrow), treatment with polyclonal 50 μg anti-BMP1.3 (FIG. 2C), 2 h after 3rd treatment (FIG. 2D), compared WT mouse (green arrow) and treated DyW−/− (red arrow) in 5th week of life (FIG. 2E); treated DyW−/− in 5th week of life (FIG. 2F). The mouse carrying homozygous mutation for laminin α2 gene (Lama2 Dy-W) is smaller in size when compared with the wild type sibling, less active, and has impaired motion. After treatment with polyclonal anti-BMP1.3 antibody (50 μg/kg), its activity was improved after the 1st dose, and mobility was even more improved after subsequent two doses, and it lived for 5 weeks, whereas homozygous mice usually die at 4 weeks, which is a life extension of 20% with this arbitrary therapeutic approach. Interestingly, we also found an increase in BMP1.3 transcripts in liver, muscle and kidney tissues of homozygous DyW mice as compared to heterozygous or age matched wild type aged 5 weeks (FIG. 3). The DyW mice are bred and experiments were conducted following standardized procedures for CMD-related mice and their phenotypic characteristics, specifically in survival, muscle weight, body weight and hind limb paralysis. Thus, we were able to establish credentialing that BMP1.3 is elevated in the skeletal muscle of DyW mice.

Mice homozygous for the mutation in the laminin-α2 gene (DyW mouse strain), after treatment with polyclonal anti-BMP1.3 antibody, showed surprising improvement of mobility and overall activity and their lifespan was prolonged up to 5 weeks (it usually lasts for 2-4 weeks in DyW mice, depending on the genetic background and food consumption.

FIG. 2D shows the stunning improving of DyW mice mobility, front leg upward extension on the cage wall, and hind leg use to increase speed and jump, improving thus significantly the quality of life and, importantly, longevity by 25% of the usual life span of 2-4 weeks due to their specific genetic background. This has not been previously observed by any previously reported therapies. Thus, treatment of DyW mice with a specific BMP1.3 polyclonal antibody had a stunning effect on the quality of movement, muscle volume and longevity.

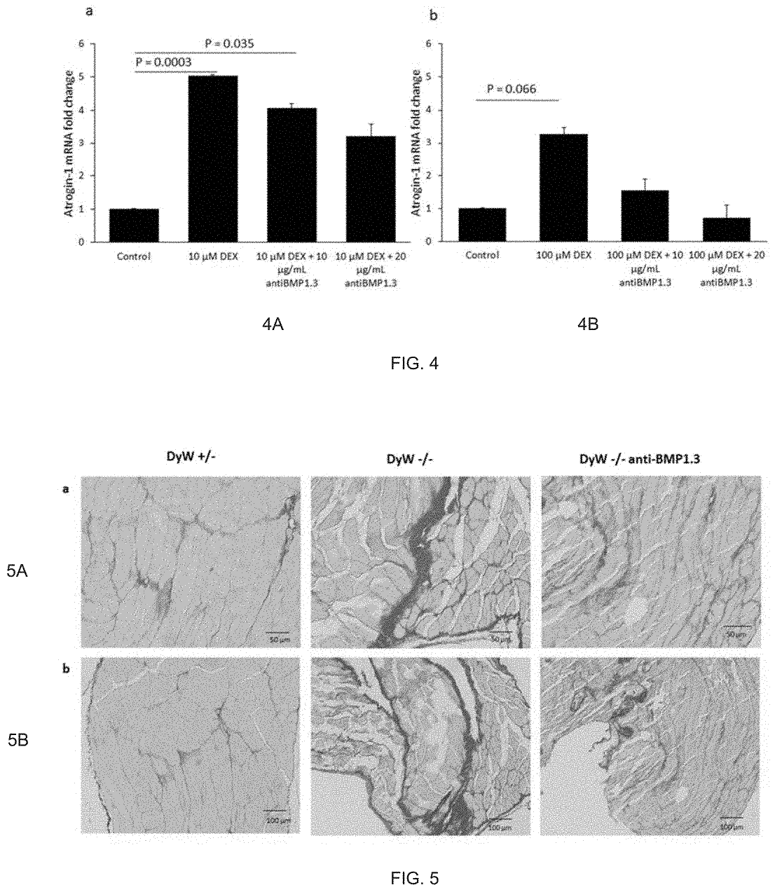

It has been previously reported that inflammation and fibrosis are very early signatures of MDC1A pathology, and we noticed upregulation of collagen in muscle tissues isolated from DyW mice (FIG. 4). It is therefore possible that the BMP1.3 molecule could have played a critical role in collagen processing in DyW pathology thus reducing muscle fibrosis. A correlation with an enhanced BMP1.3 expression in DyW mice and its inhibition with anti-BMP1.3 antibody leading to amelioration of fibrogenic pathology associated with MDC1A.

We found an unexpectedly high BMP1.3 expression in skeletal muscles of CDM (DyW) mice which is worth exploring for its function in congenital muscular dystrophy since there is no therapy to rescue or extend life of these children. DyW mice and patients with LAMA2 related muscular dystrophy have a chronic dysregulated remodeling of the myomatrix (Accorsi et al. Frontiers Molec Neurosci 2020). Our findings in CMD mice is comparable to the effects of anti-BMP1 antibodies in CKD model (5/6 nephrectomized (Nx) rats) and AMI (cardiac ischemia and repurfusion) model in prevention and restoration mode. Further evaluation of the effects of anti-BMP1.3 antibodies showed it has substantially reduced renal interstitial and cardiac muscle fibrosis and improved its function respectively. This observation was extended in hepatic fibrosis models and cutaneous scarring models as well. It is worth to mention that BMP1.3 level is elevated 3-fold in patients with CKD and AMI as compared to age matched healthy individuals.

For the first time, it has been observed that BMP1.3 gene expression was highly enhanced in the skeletal muscles of mice with congenital muscular dystrophy (CMD), while administration of anti-BMP1.3 antibody improved the mobility, increased muscle volume and survival of CMD-DyW mice. We claim that BMP1.3 inhibition represents a novel therapeutic strategy in reversing the progression of CMD.

### METHODOLOGY AND EXAMPLES OF THE INVENTION

Generation of Mouse and Human Anti-BMP1.3 Antibodies with Several Epitopes which may Span Across the Structure of Catalytic and Possible Non-Catalytic Domains

Antibodies against the BMP1.3 non-catalytic domain or alternatively against BMP1.3 CUB-3 and CUB-4 domains involved in binding to its substrates may inhibit the function of BMP1.3. To generate and characterize those antibodies one can produce synthetic BMP1.3 peptides or produce the entire rhBMP1.3 or domains of BMP1.3. We have addressed BMP1.3 protein expression issue by two approaches. The first approach is to disable the protease activity by site directed mutagenesis of the catalytic region in the BMP1.3. While the protease activity is now lacking one still obtains a protein that is useful for binding studies and moreover for selecting monoclonal antibodies. The second approach is to produce the BMP1.3 in transient expression system such as HEK 293 cells and possibly in the Baculovirus system. It is also most useful to control the expression using a genetic switch (Tet-switch) to prevent expression in the early stage of plasmid transfection.

We will use a human phage display antibody library which contains 10¹⁰ different specificities, whereby the format is display of Fab fragments as gene III fusions. This phage library can yield an abundance of useful BMP1.3 antibodies which need to be characterized regarding the epitope and affinities. After identification of Fab antibodies binding to BMP1.3, which are produced in E. coli, the resulting Fab molecules will finally be reformatted by insertion into mammalian expression vectors to secrete of entire IgG molecules containing the entire constant regions.

Screen for Safety and Efficacy in Preclinical Models of Congenital Muscular Dystrophy and Related Duchenne Muscular Dystrophy—Define Dose and Dosing Regiments

The levels of BMP1.3 both at transcript and protein levels in DyW muscle as well as in the serum are determined. An optimum dose for treatment with specific anti-BMP1.3 antibodies will provide survival benefit when started in 1st postnatal week. Whether the optimum dose with treatment in 1st week postnatal for 6 weeks will make an improvement in survival, grip strength, locomotory function, weight, histopathology (number of fibers, fiber size), the next steps will be determined.

Magnetic resonance imaging, as a robust measure of muscle mass, contractile area, inflammation, and fibrosis, will be used in all mouse experiments. Intrinsic muscle properties will be measured by electrical impedance myography.

Mobility of DyW mice will be recorded via a computer-controlled activity assay and measure the distance traveled and increase in vertical breaks indicative of increased use of hind limbs during the assay. Six-week studies will be performed on cohorts of DyW mice and their WT controls to optimize the dosage of anti-BMP1.3 treatment. Power analysis predicts, with an expectation to achieve a difference of 15-20% in the measured variables between the treated and untreated group, and to reach a significance level of 0.05 at 95% confidence level, 12 animals per cohort will be used.

Examine the Substrate Specificity and the Mechanism of BMP1.3 Action as it Relates to the Downstream Signaling and Pathways Associated with Inflammation, Fibrosis and Regeneration

Determination of substrate specificity of BMP1 isoform will be initially performed using total cell extracts of HEK293 cells, H4IIE hepatocytes and targeted myocytes treated with different concentration of rBMP1.1 and rBMP1.3. Cleavage sites will be identified by mass spectrometry using the COFRADIC technology in our suitable equipped laboratory (LTQ Velos Orbitrap (Thermo Scientific) and an ultrafleXtreme™ III MALDI-TOF/TOF (Bruker) mass spectrometers). To confirm the site of increased BMP1.3 expression, in vitro cultures of primary epithelial and endothelial cells, fibroblasts, pericytes, hepatocytes and myocytes isolated from healthy and pathological tissue samples will be established and analyzed by immunofluorescence and FACS. Also, expression of BMP1.3 in human kidney, liver and heart disease sections will be evaluated at the RNA (RT-PCR) and protein level (Western blot and ELISA). The mechanisms of BMP1.3 action will be characterized. Study of the known targets of BMP1.3 (Laminin, collagen, decorin, BMP2, -4 and -7, TGF-β1, CTGF, LTBP transglutaminase and MMPs) and effectiveness on TGF-β1 activation at the systemic and receptor level will be performed. Proteins will be quantified in the kidney, liver, heart and peripheral muscle. In addition, mRNA from these tissues will be profiled to identify responsive cellular networks in tissues. Comparing normal and genetically modified mice the influence of different BMP1.3 levels on the various fibrotic processes will be determined. In experiments to elucidate the BMP1.3 function in acute myocardial infarction (AMI) based on the substrate specificities, new fluorogenic assays for in vitro work will be developed. In in vitro experiments cardiac fibroblasts will be treated with BMP1.3 protein and anti-BMP1.3 antibodies will be tested and both the collagen and αSMA amounts will be determined, as compared to untreated cells. To examine the potential early beneficial effects of BMP1.3 antibodies, fibroblasts and cardiomyocytes will be cultured in hypoxic chamber, which resembles AMI state. A number of both fibroblasts and cardiomyocytes which will undergo apoptosis will be explored. Expression of selected genes in cardiomyocyte and fibroblast culture, including Bmp2, Bmp5, Col1α1, Tgfβ1, rAgrin and Id1-4 expression in fibroblasts will be tested.

To determine the role of BMP1.3 isoform, primary cell cultures will be treated with different concentrations of rBMP1 isoforms and αBMP1.3 mAbs to evaluate: gene expression of TGF-β, RASAL1, FGF-2, PDGF, DNMT1, COL1A1, αSMA molecule, synthesis/release of growth factors and ECM components (TGF-β, FGF-2, COL1A1) involved in fibrogenesis and functional parameters of cell migration, proliferation and vitality. By transcriptomic and miRNA analyses and with silencing BMP1.3 transcripts in those cells, as well as in the cells from transgenic mice overexpressing BMP1.3, we will try to explain the effect of the lack of gene product on induction of molecular and functional changes.

Acute myocardial infarction (AMI) mouse model will be used to assess the safety and efficiency of the selected anti-BMP1.3 antibodies as a second rodent model with a strong inflammatory and fibrotic component. BMP1.3 bioactivity on those BMP molecules in which prodomains (pd) confer latency to its growth factor domain (like BMP10) will be tested. AMI will be induced by ligation of the left coronary artery in two months old CD-1 mice. The apical region of the heart, fixed in formalin solution will be embedded in paraffin and four micrometer sections will be stained with H&E and observed microscopically. Sirius red staining of heart sections will be performed to enable assessment of ratio of fibrosis and quantified by using S-form software. Additional immunohistochemistry will be done on selected heart sections to detect expression of fibrosis related molecules.

Humanize the Best BMP1.3 Antibody to Perform Safety and Efficacy Testing in Preclinical Development Including Non-Human Primates

Creating an antibody against the catalytic domain which has been found to be relatively promiscuous, indicates that the specificity should be conferred by the non-catalytic domains. Both mTLD and mTLL-1 form dimers, in such a way that the catalytic domain is relatively hidden, suggesting a substrate exclusion mechanism for the control of substrate specificity.

mAbs will be humanized on the gene level and then produced by transfection of the entirely synthetic genes into HEK 293 and CHO cells. Humanized Abs will then be produced from resulting stable transfectoma cell lines. In the course of the project, assays for measuring BMP1.3 in biological fluids will be improved, which will enable a better understanding of its use as a biomarker of CKD, HF and AMI with development of appropriate tools for its measurement.

For mAbs, the most relevant safety assessment species is the Cynomolgus monkey (Macaca fascicularis) and this species cross-reacted with the majority of currently approved mAbs. These animals represent a requirement for the purposes of accurate safety assessment, and they will be used in preclinical testing by an EU CRO according to ICH safety testing guidelines (ICH M3 (R2); ICH S9, 2009 and ICH S6 (R1)).

Obtain Approvals for Unblinded Uncontrolled Studies in a Few (10-12) Volunteers to Assess Safety in Humans Using the Selected Antibody and for the Future Clinical Trial in Patients with Orphan Congenital Muscle Dystrophy

For approval of clinical trials, we will produce the antibody in a stable cell line in a GMP manufacturing facility with full documentation. We will employ analytical procedures for quality control, define release criteria to be met, demonstrate three successful production batches, sterility testing, viral spiking, perform stability studies for at least 3 months, and finally, all details will be approved by a qualified person. We will submit all applicable documentation to local regulatory authorities in order to obtain clinical trial approvals.

The production of monoclonal antibodies is highly efficient in engineered CHO cells lines and reaches very high levels while relying on the single-use technology in wave reactors or stirred tanks which we operate in our clean-room facility. We have all fermentation, purification and analytical capabilities in house, as well as experience and business relationships with several CROs as well as substantial experience in fulfilling regulatory requirements of clinical trials. Following IMPD submission and receiving approval from the regulatory agency in Croatia unblinded uncontrolled studies in 10-12 volunteers to assess safety in humans using the selected antibody will be performed to create conditions to facilitate clinical trials in CMD orphan indications in approximately 10 LAMA2 CMD patients (17 available in Croatia) and, if successful, receive the fast track data analysis and approval by EMA.

### EXAMPLE

The features and advantages of the invention are more fully shown by the following non-limiting example.

Breeding and Pathophysiology of Homozygous CMD (DyW) Mice in House

To support translatability and credentialing of results from mouse to human, we will use several mouse models, in particular mice with LAMA2-CMD mutation, but due to different genetic background, their phenotypic characteristics are very variable, specifically in survival, muscle weight, body weight and hind limb paralysis. The DyW mice are bred and experiments were conducted following standardized procedures for CMD-related mice, which include analyses of the genetic background, food composition, careful estimation of the minimal sample sizes required to obtain sufficient statistical power to improve consistency of results and translatability of efficacy studies. Standardized protocols are adjusted according to www.curecmd.org.

Evaluation of Anti-BMP1.3 Antibody Against DyW Mice—Translatability

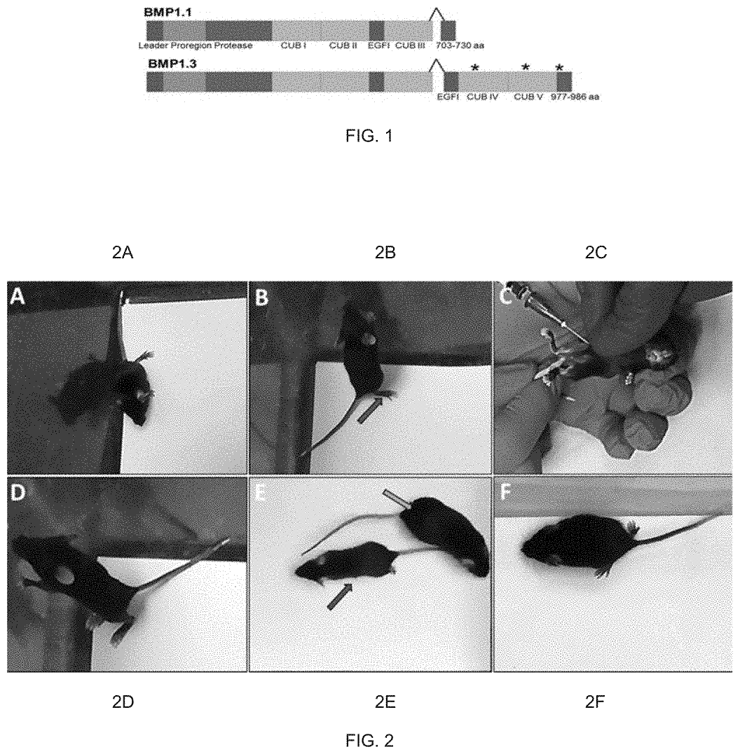

Utilizing LC-MS, we identified BMP1.3 isoform of the Bmp1 gene (FIG. 1) in the plasma of healthy volunteers and patients with CKD, HF and AMI, which was the first direct evidence of its natural existence on the protein level.

Mice homozygous for the mutation in the laminin-α2 gene (DyW mouse strain), after treatment with polyclonal anti-BMP1.3 antibody, showed surprising improvement of mobility and overall activity and their lifespan was prolonged up to 5 weeks (it usually lasts for 2-4 weeks in DyW mice, depending on the genetic background and food consumption as priorly evaluated by Willmann et al. FIG. 2 shows the stunning improving of DyW mice mobility, front leg upward extension on the cage wall, and hind leg use to increase speed and jump, improving thus significantly the quality of life and, importantly, longevity by 25% of the usual life span of 4 weeks due to their specific genetic background. Thus, treatment of DyW mice with a specific BMP1.3 polyclonal antibody had a stunning effect on the quality of movement, muscle volume and longevity.

Ex vivo analyses showed an increased BMP1.3 expression in muscles of DyW mice. Interestingly, we also found an increase in BMP1 and BMP1.3 transcripts in liver, muscle and kidney tissues of DyW mice aged 5 weeks (FIG. 3). Expression of TGF-β and Smad genes was also upregulated in DyW mice, as well as BMP2 and BMP5 (FIG. 3). In addition, we tested the common Smad4 and TGF-β specific Smad2 and -3 and TGF-β, BMP2 and -5 expression in homozygous DyW mice as compared to heterozygote animals and found extremely increased expression of all genes, indicting a very disrupted system of ECM and signaling TGF-β family members (FIG. 3).

In parallel, treatment of myoblasts with BMP1.3 antibodies had a significant negative effect on the progress of atrophy induced by addition of 10 μM dexamethasone, which was demonstrated by the decreased expression of atrophy marker Atrogin-1 (FIG. 4).

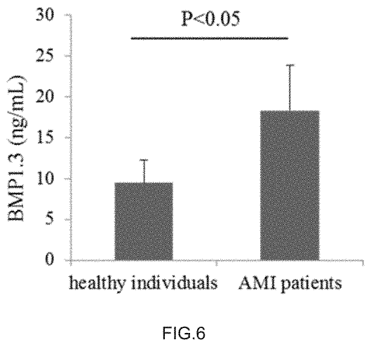

It has been previously reported that inflammation and fibrosis are a very early signature of MDC1A pathology and we noticed significant increase in collagen staining in muscle tissues isolated from DyW mice (FIG. 5). It is obvious that BMP1.3 plays a critical role in collagen processing in DyW pathology. BMP1.3 inhibition leads to amelioration of fibrogenic pathology associated with MDC1A in DyW mouse model confirming its translatability.

BMP1.3 Levels in AMI Patients (Credentialing) and Effects of Anti-BMP1.3 Antibody in AMI Models (Translatability)

By applying an ELISA assay, we analyzed blood samples from healthy humans and patients with AMI. Patients with AMI have significantly higher levels of circulating BMP1.3 protein in comparison to healthy individuals (FIG. 6). Increased levels of BMP1.3 were observed also in the plasma of patients with CKD. These findings provided the rationale and credentialing for testing the efficacy of BMP1.3 inhibition with monoclonal antibodies in reducing fibrosis deposition in various organs. Based on the evidence that BMP1.3 inhibition in the kidney successfully prevents excessive fibrosis, we aimed to explore a possible mechanism and therapeutic applicability of BMP1.3 inhibition in rodent model of AMI.

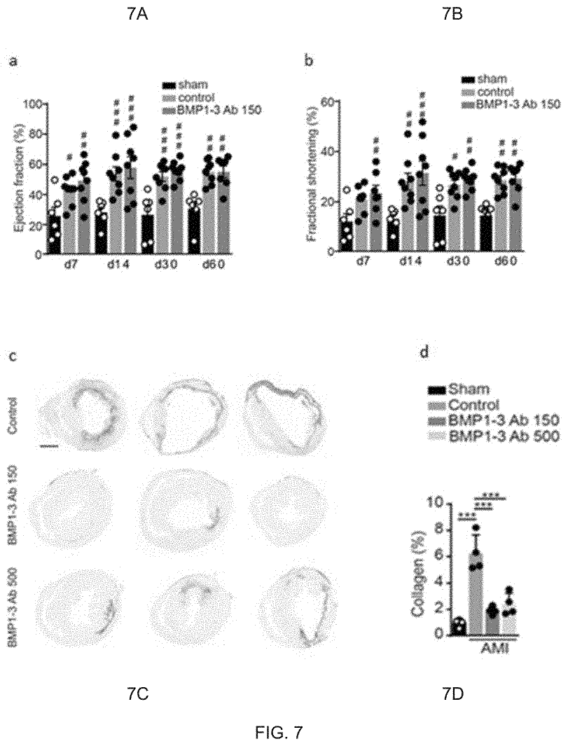

The peptide used (aa759-772; CTSPNWPDKYPSKKE) for producing BMP1.3 antibody and its use in a rat heart coronary ligation model resulted in a significant improvement of functional heart parameters. FIG. 7 illustrates BMP1.3 polyclonal antibody reduced fibrosis and preserved ejection fraction in rodent models of AMI. Therapeutic efficacy of BMP1.3 antibody was tested in a mouse model of AMI. Surgical ligation of LAD coronary artery was performed in mice to produce AMI which is mainly restricted to left ventricle myocardium. The antibody was administered at low (150 μg/kg) and high (500 μg/kg) dose. Follow-up of mice was enabled with echocardiographic examination on pre-defined time points. Mice (n=10 per group) were followed for 2 months. (A) Quantification of ejection fraction at 7, 14, 30 and 60 days after myocardial infarction. (B) Quantification of fractional shortening at 7, 14, 30 and 60 days after myocardial infarction. (C) Histology and histomorphometric analyses of mice hearts were performed after 2 months. As expected, control rats subjected to AMI and without treatment develop significant thickening of left ventricle and vast deposition of fibrotic tissue. (D) Scar formation in animals treated with both low and high dose of BMP1.3 antibody.

These results with BMP1.3 inhibition in rodent models of AMI confirmed the role of BMP1.3 in preventing inflammation and fibrosis thus supporting current study in CMD models and potential first-class novel global therapy for humans with MDCA1 due to appropriate translatability and strong credentialing as critical premises for successful drug development for patients.

The following references are hereby incorporated by reference:
1. L. Grgurevic, B. Macek, D. R. Healy, A. L. Brault, I. Erjavec, A. Cipcic, I. Grgurevic, D. Rogic, K. Galesic, J. Brkljacic, R. Stern-Padovan, V. M. Paralkar, S. Vukicevic. Circulating bone morphogenetic protein 1-3 isoform increases renal fibrosis, J Am Soc Nephrol. 22 (2011):681-692.
2. L. Grgurevic, I. Erjavec, I. Grgurevic, I. Dumic-Cule, J. Brkljacic, D. Verbanac, M. Matijasic, H. C. Paljetak, R. Novak, M. Plecko, J. Bubic-Spoljar, D. Rogic, V. Kufner, M. Pauk, T. Bordukalo-Niksic, S. Vukicevic. Systemic inhibition of BMP1-3 decreases progression of CCl4-induced liver fibrosis in rats, Growth Factors. 35 (2017):201-215.
3. I. Dumic-Cule, V. Martinelli, V. Kufner, S. Moimas, B. Brkljacic, D. Delic-Brkljacic, M. Milosevic, M. Giacca, S. Zacchigna, S. Vukicevic. Bone Morphogenetic Protein 1-3 inhibition supports cardiomyocyte survival and decreased collagen deposition after acute myocardial infarction in rodents, Proc Natl Acad Sci USA. in press (2020).
4. C. Chiari, L. Grgurevic, T. Bordukalo-Niksic, H. Oppermann, A. Valentinitsch, E. Nemecek, K. Staats, M. Schreiner, C. Trost, A. Kolb, F. Kainberger, S. Pehar, M. Milosevic, S. Martinovic, M. Peric, T. K. Sampath, S. Vukicevic, R. Windhager. Recombinant human BMP6 applied within autologous blood coagulum accelerates bone healing: randomized controlled trial in high tibial osteotomy patients J Bone Miner Res.10.1002/jbmr.4107(2020).
5. L. Grgurevic, I. Erjavec, M. Gupta, M. Pecin, T. Bordukalo-Niksic, N. Stokovic, D. Vnuk, V. Farkas, H. Capak, M. Milosevic, J. B. Spoljar, M. Peric, M. Vuckovic, D. Maticic, R. Windhager, H. Oppermann, T. K. Sampath, S. Vukicevic. Autologous blood coagulum containing rhBMP6 induces new bone formation to promote anterior lumbar interbody fusion (ALIF) and posterolateral lumbar fusion (PLF) of spine in sheep, Bone.10.1016/j.bone.2020.115448(2020):115448.
6. J. S. Huston, D. Levinson, M. Mudgett-Hunter, M. S. Tai, J. Novotny, M. N. Margolies, R. J. Ridge, R. E. Bruccoleri, E. Haber, R. Crea, et al. Protein engineering of antibody binding sites: recovery of specific activity in an anti-digoxin single-chain Fv analogue produced in Escherichia coli, Proc Natl Acad Sci USA. 85 (1988):5879-5883.
7. Q. Nguyen, K. R. Q. Lim, T. Yokota. Current understanding and treatment of cardiac and skeletal muscle pathology in laminin-alpha2 chain-deficient congenital muscular dystrophy, Appl Clin Genet. 12 (2019):113-130.
8. A. M. Connolly, R. M. Keeling, S. Mehta, A. Pestronk, J. R. Sanes. Three mouse models of muscular dystrophy: the natural history of strength and fatigue in dystrophin-, dystrophin/utrophin-, and laminin alpha2-deficient mice, Neuromuscul Disord. 11 (2001):703-712.
9. J. E. Rooney, P. B. Gurpur, D. J. Burkin. Laminin-111 protein therapy prevents muscle disease in the mdx mouse model for Duchenne muscular dystrophy, Proc Natl Acad Sci USA. 106 (2009):7991-7996.
10. F. M. Tome, T. Evangelista, A. Leclerc, Y. Sunada, E. Manole, B. Estournet, A. Barois, K. P. Campbell, M. Fardeau. Congenital muscular dystrophy with merosin deficiency, C R Acad Sci III. 317 (1994):351-357.
11. J. Philpot, A. Bagnall, C. King, V. Dubowitz, F. Muntoni. Feeding problems in merosin deficient congenital muscular dystrophy, Arch Dis Child. 80 (1999):542-547.
12. R. Vohra, A. Accorsi, A. Kumar, G. Walter, M. Girgenrath. Magnetic Resonance Imaging Is Sensitive to Pathological Amelioration in a Model for Laminin-Deficient Congenital Muscular Dystrophy (MDC1A), PLoS One. 10 (2015):e0138254.
13. M. Girgenrath, J. A. Dominov, C. A. Kostek, J. B. Miller. Inhibition of apoptosis improves outcome in a model of congenital muscular dystrophy, J Clin Invest. 114 (2004):1635-1639.
14. W. Kuang, H. Xu, P. H. Vachon, L. Liu, F. Loechel, U. M. Wewer, E. Engvall. Merosin-deficient congenital muscular dystrophy. Partial genetic correction in two mouse models, J Clin Invest. 102 (1998):844-852.
15. Y. Miyagoe, K. Hanaoka, I. Nonaka, M. Hayasaka, Y. Nabeshima, K. Arahata, Y. Nabeshima, S. Takeda. Laminin alpha2 chain-null mutant mice by targeted disruption of the Lama2 gene: a new model of merosin (laminin 2)-deficient congenital muscular dystrophy, FEBS Lett. 415 (1997):33-39.
16. T. Mehuron, A. Kumar, L. Duarte, J. Yamauchi, A. Accorsi, M. Girgenrath. Dysregulation of matricellular proteins is an early signature of pathology in laminin-deficient muscular dystrophy, Skelet Muscle. 4 (2014):14.
17. A. Accorsi, T. Mehuron, A. Kumar, Y. Rhee, M. Girgenrath. Integrin dysregulation as a possible driver of matrix remodeling in Laminin-deficient congenital muscular dystrophy (MDC1A), J Neuromuscul Dis. 2 (2015):51-61.
18. E. M. Clement, L. Feng, R. Mein, C. A. Sewry, S. A. Robb, A. Y. Manzur, E. Mercuri, C. Godfrey, T. Cullup, S. Abbs, F. Muntoni. Relative frequency of congenital muscular dystrophy subtypes: analysis of the UK diagnostic service 2001-2008, Neuromuscul Disord. 22 (2012):522-527.
19. F. L. Norwood, C. Harling, P. F. Chinnery, M. Eagle, K. Bushby, V. Straub. Prevalence of genetic muscle disease in Northern England: in-depth analysis of a muscle clinic population, Brain. 132 (2009):3175-3186.
20. F. Geranmayeh, E. Clement, L. H. Feng, C. Sewry, J. Pagan, R. Mein, S. Abbs, L. Brueton, A. M. Childs, H. Jungbluth, C. G. De Goede, B. Lynch, J. P. Lin, G. Chow, C. Sousa, O. O'Mahony, A. Majumdar, V. Straub, K. Bushby, F. Muntoni. Genotype-phenotype correlation in a large population of muscular dystrophy patients with LAMA2 mutations, Neuromuscul Disord. 20 (2010):241-250.
21. H. Colognato, P. D. Yurchenco. The laminin alpha2 expressed by dystrophic dy(2J) mice is defective in its ability to form polymers, Curr Biol. 9 (1999):1327-1330.
22. H. Xu, X. R. Wu, U. M. Wewer, E. Engvall. Murine muscular dystrophy caused by a mutation in the laminin alpha 2 (Lama2) gene, Nat Genet. 8 (1994):297-302.
23. J. R. Reinhard, S. Lin, K. K. McKee, S. Meinen, S. C. Crosson, M. Sury, S. Hobbs, G. Maier, P. D. Yurchenco, M. A. Ruegg. Linker proteins restore basement membrane and correct LAMA2-related muscular dystrophy in mice, Sci Transl Med. 9 (2017).
24. S. Vukicevic, F. P. Luyten, H. K. Kleinman, A. H. Reddi. Differentiation of canalicular cell processes in bone cells by basement membrane matrix components: regulation by discrete domains of laminin, Cell. 63 (1990):437-445.
25. V. M. Paralkar, S. Vukicevic, A. H. Reddi. Transforming growth factor beta type 1 binds to collagen IV of basement membrane matrix: implications for development, Dev Biol. 143 (1991):303-308.
26. A. Accorsi, A. Kumar, Y. Rhee, A. Miller, M. Girgenrath. IGF-1/GH axis enhances losartan treatment in Lama2-related muscular dystrophy, Hum Mol Genet. 25 (2016):4624-4634.
27. S. Meinen, S. Lin, M. A. Ruegg. Angiotensin II type 1 receptor antagonists alleviate muscle pathology in the mouse model for laminin-alpha2-deficient congenital muscular dystrophy (MDC1A), Skelet Muscle. 2 (2012):18.
28. P. K. Siegl, R. S. Chang, N. B. Mantlo, P. K. Chakravarty, D. L. Ondeyka, W. J. Greenlee, A. A. Patchett, C. S. Sweet, V. J. Lotti. In vivo pharmacology of L-158,809, a new highly potent and selective nonpeptide angiotensin II receptor antagonist, J Pharmacol Exp Ther. 262 (1992):139-144.
29. M. Erb, S. Meinen, P. Barzaghi, L. T. Sumanovski, I. Courdier-Fruh, M. A. Ruegg, T. Meier. Omigapil ameliorates the pathology of muscle dystrophy caused by laminin-alpha2 deficiency, J Pharmacol Exp Ther. 331 (2009):787-795.
30. W. A. Grasser, I. Orlic, F. Borovecki, K. A. Riccardi, P. Simic, S. Vukicevic, V. M. Paralkar. BMP-6 exerts its osteoinductive effect through activation of IGF-I and EGF pathways, Int Orthop. 31 (2007):759-765.
31. P. Barraza-Flores, H. J. Hermann, C. R. Bates, T. G. Allen, T. T. Grunert, D. J. Burkin. Human laminin-111 and laminin-211 protein therapy prevents muscle disease progression in an immunodeficient mouse model of LAMA2-CMD, Skelet Muscle. 10 (2020):18.
32. C. C. Fontes-Oliveira, M. S. O. B, Z. Korner, M. H. V, M. Durbeej. Effects of metformin on congenital muscular dystrophy type 1A disease progression in mice: a gender impact study, Sci Rep. 8 (2018):16302.
33. H. Dorai, J. E. McCartney, R. M. Hudziak, M. S. Tai, A. A. Laminet, L. L. Houston, J. S. Huston, H. Oppermann. Mammalian cell expression of single-chain Fv (sFv) antibody proteins and their C-terminal fusions with interleukin-2 and other effector domains, Biotechnology (N Y). 12 (1994):890-897.
34. E. Ozkaynak, D. C. Rueger, E. A. Drier, C. Corbett, R. J. Ridge, T. K. Sampath, H. Oppermann. OP-1 cDNA encodes an osteogenic protein in the TGF-beta family, EMBO J. 9 (1990):2085-2093.
35. E. Ozkaynak, P. N. Schnegelsberg, D. F. Jin, G. M. Clifford, F. D. Warren, E. A. Drier, H. Oppermann. Osteogenic protein-2. A new member of the transforming growth factor-beta superfamily expressed early in embryogenesis, J Biol Chem. 267 (1992):25220-25227.
36. R. Willmann, H. Gordish-Dressman, S. Meinen, M. A. Ruegg, Q. Yu, K. Nagaraju, A. Kumar, M. Girgenrath, C. B. M. Coffey, V. Cruz, P. M. Van Ry, L. Bogdanik, C. Lutz, A. Rutkowski, D. J. Burkin. Improving Reproducibility of Phenotypic Assessments in the DyW Mouse Model of Lam inin-alpha2 Related Congenital Muscular Dystrophy, J Neuromuscul Dis. 4 (2017):115-126.
37. S. Pasteuning-Vuhman, K. Putker, C. L. Tanganyika-de Winter, J. W. Boertje-van der Meulen, L. van Vliet, M. Overzier, J. J. Plomp, A. Aartsma-Rus, M. van Putten. Natural disease history of the dy2J mouse model of laminin alpha2 (merosin)-deficient congenital muscular dystrophy, PLoS One. 13 (2018):e0197388.
38. M. D. Gomes, S. H. Lecker, R. T. Jagoe, A. Navon, A. L. Goldberg. Atrogin-1, a muscle-specific F-box protein highly expressed during muscle atrophy, Proc Natl Acad Sci USA. 98 (2001):14440-14445.
39. P. Sicinski, Y. Geng, A. S. Ryder-Cook, E. A. Barnard, M. G. Darlison, P. J. Barnard. The molecular basis of muscular dystrophy in the mdx mouse: a point mutation, Science. 244 (1989):1578-1580.
40. M. L. Boland, S. Oldham, B. B. Boland, S. Will, J. M. Lapointe, S. Guionaud, C. J. Rhodes, J. L. Trevaskis. Nonalcoholic steatohepatitis severity is defined by a failure in compensatory antioxidant capacity in the setting of mitochondrial dysfunction, World J Gastroenterol. 24 (2018):1748-1765.
41. D. S. Rice, T. Curran. Mutant mice with scrambled brains: understanding the signaling pathways that control cell positioning in the CNS, Genes Dev. 13 (1999):2758-2773.
42. K. Verhoef, G. Marzio, W. Hillen, H. Bujard, B. Berkhout. Strict control of human immunodeficiency virus type 1 replication by a genetic switch: Tet for Tat, J Virol. 75 (2001):979-987.
43. C. H. Hakim, A. Mijailovic, T. B. Lessa, J. R. Coates, C. Shin, S. B. Rutkove, D. Duan. Non-invasive evaluation of muscle disease in the canine model of Duchenne muscular dystrophy by electrical impedance myography, PLoS One. 12 (2017):e0173557.
44. J. E. Rooney, J. R. Knapp, B. L. Hodges, R. D. Wuebbles, D. J. Burkin. Laminin-111 protein therapy reduces muscle pathology and improves viability of a mouse model of merosin-deficient congenital muscular dystrophy, Am J Pathol. 180 (2012):1593-1602.
45. K. Gevaert, M. Goethals, L. Martens, J. Van Damme, A. Staes, G. R. Thomas, J. Vandekerckhove. Exploring proteomes and analyzing protein processing by mass spectrometric identification of sorted N-terminal peptides, Nat Biotechnol. 21 (2003):566-569.
46. C. Wermter, M. Howel, V. Hintze, B. Bombosch, K. Aufenvenne, I. Yiallouros, W. Stocker. The protease domain of procollagen C-proteinase (BMP1) lacks substrate selectivity, which is conferred by non-proteolytic domains, Biol Chem. 388 (2007):513-521.
47. K. Iwasaki, Y. Uno, M. Utoh, H. Yamazaki. Importance of cynomolgus monkeys in development of monoclonal antibody drugs, Drug Metab Pharmacokinet. 34 (2019):55-63.
48. H. Weber, E. Berge, J. Finch, P. Heidt, F. J. Kaup, G. Perretta, B. Verschuere, S. Wolfensohn, F. W. G. N. H. P. Hlth. Health monitoring of non-human primate colonies—Recommendations of the Federation of European Laboratory Animal Science Associations (FELASA) Working Group on non-human primate health accepted by the FELASA Board of Management, 21 Nov. 1998, Laboratory Animals. 33 (1999):3-18.

What is claimed is:

1. A method of treating and delaying the progression of muscular dystrophy comprising of administering to an individual in need thereof a pharmaceutical composition comprising an antibody that binds to BMP1.3 non-catalytic domain that contains either CUB-3 or CUB-4, or both CUB-3 and CUB-4, or EGF-2, or both CUB-3 and CUB-4 and EGF-2 and at the C-terminal region.

2. The method according to claim 1, wherein the individual suffers from muscular dystrophy selected from the group consisting of congenital muscular dystrophy (CMD), Duchenne muscular dystrophy (DMD), Myotonic muscular dystrophy, Becker muscular dystrophy, Limb-girdle muscular dystrophy, Facioscapulohumeral muscular dystrophy, Oculopharyngeal muscular dystrophy, Distal muscular dystrophy, and Emery-Dreifuss muscular dystrophy.

3. The method according to claim 2, wherein said individual is onset of birth to 2-years following birth.

4. The method according to claim 2, wherein said individual is within 2-10 years of birth.

5. The method according to claim 2, wherein said individual is juvenile.

6. The method according to claim 2, wherein said individual is adult.

7. The method according to claim 2, wherein the Congenital Muscular Dystrophy (CMD) is associated with collagen VI related myopathies, merosin deficient CMDs [MDCs], laminin a2 [LAMA2]-related CMDs, and MDC1A; and a-dystroglycan-related MDs.

8. The method according to claim 1, wherein the antibody that binds to BMP1.3 non-catalytic domain that contain either CUB 3 or CUB4 or both CUB 3 and CUB4 or EGF-2 at the C-terminal region is a humanized antibody.

9. The method according to claim 1, wherein administration of the pharmaceutical composition reduces muscle fibrosis, increases muscle mass, preserves muscle contractility, or preserves muscle strength in the indivdual.

10. The method according to claim 1, wherein administration of the pharmaceutical composition protects against muscle contraction-induced injury in the individual.

11. The method of claim 1, wherein the pharmaceutical composition is formulated for subcutaneous, intramuscular, or intravenous administration.

12. The method of claim 1, wherein the pharmaceutical composition is administered at a low dose.

13. The method of claim 1, wherein the pharmaceutical composition is administered at a dose between 0.1 mg/kg per day to 10 mg/kg per day.

14. The method of claim 1, wherein the pharmaceutical composition is administered daily, from one to three times weekly, weekly, or from one to two times a month or once in 3 months.

15. A method of producing an antibody that binds to BMP1.3 non-catalytic domain that contains either CUB-3 or CUB-4, or both CUB-3 and CUB-4, or EGF-2, or both CUB-3 and CUB-4 and EGF-2 and at the C-terminal region.

16. A pharmaceutical composition comprising an effective amount of an antibody that binds to BMP1.3 non-catalytic domain that contains either CUB-3 or CUB-4 or both CUB-3 and CUB-4 or EGF-2 or both CUB-3 and CUB-4 and EGF-2 and at the C-terminal region for use in treating and delaying the progression of muscular dystrophy in an individual.

17. The pharmaceutical composition of claim 16 for use in treating and delaying of congenital muscular dystrophy (CMD), Duchenne muscular dystrophy (DMD), Myotonic muscular dystrophy, Becker muscular dystrophy, Limb-girdle muscular dystrophy, Facioscapulohumeral muscular dystrophy, Oculopharyngeal muscular dystrophy, Distal muscular dystrophy, and Emery-Dreifuss muscular dystrophy.

18. An antibody that binds to BMP1.3 non-catalytic domain that contains either CUB-3 or CUB-4 or both CUB-3 and CUB-4 or EGF-2 or both CUB-3 and CUB-4 and EGF-2 and at the C-terminal region for use in treating and delaying the progression of muscular dystrophy in an individual.

19. The antibody of claim 18, wherein muscular dystrophy is congenital muscular dystrophy (CMD), Duchenne muscular dystrophy (DMD), Myotonic muscular dystrophy, Becker muscular dystrophy, Limb-girdle muscular dystrophy, Facioscapulohumeral muscular dystrophy, Oculopharyngeal muscular dystrophy, Distal muscular dystrophy, and Emery-Dreifuss muscular dystrophy.
